(19) United States
(12) Patent Application Publication
(10) Pub. No.: US 2017/0253854 A1
(43) Pub. Date: Sep. 7, 2017

(54) Use of Adipose-Derived Stem Cells for Glaucoma Treatment

(71) Applicant: University of Pittsburgh - Of the Commonwealth System of Higher Education, Pittsburgh, PA (US)

(72) Inventors: Yiqin Du, Pittsburgh, PA (US); Joel Steven Schuman, New York, NY (US)

(21) Appl. No.: 15/447,645

(22) Filed: Mar. 2, 2017

Related U.S. Application Data
(60) Provisional application No. 62/302,483, filed on Mar. 2, 2016.

(51) Int. Cl. C12N 5/079; A61K 35/30; A61K 35/28; A61K 9/00

(52) U.S. Cl. CPC C12N 5/0621; A61K 9/0048; A61K 35/30; A61K 35/28; A61K 9/0051; C12N 2502/085; C12N 2506/1384; C12N 2533/90

(57) ABSTRACT

A method of preparing a functional trabecular meshwork (TM) cell from adipose-derived stem cells is provided. Methods of treating glaucoma, implanting trabecular meshwork (TM) cells, or repairing or regenerating the aqueous outflow pathway of an eye of a patient also are provided.

### CROSS REFERENCE TO RELATED APPLICATIONS

This application claims the benefit of U.S. Provisional Patent Application No. 62/302,483. filed Mar. 2, 2016, which is incorporated herein by reference in its entirety.

The Sequence Listing associated with this application is filed in electronic format via EFS-Web and is hereby incorporated by reference into the specification in its entirety. The name of the text file containing the Sequence Listing is 6527_1701720_ST25.txt. The size of the text file is 3,110 bytes, and the text file was created on Mar. 1, 2017.

Glaucoma, the leading cause of irreversible blindness worldwide, is a progressive optic neuropathy with loss of retinal ganglion cells and axons, resulting in visual field loss. In the United States, the most common type of glaucoma is primary open angle glaucoma (POAG). Advanced age and elevated intraocular pressure (TOP) are the primary risk factors for POAG. Multiple randomized clinical trials have shown that TOP lowering is effective in delaying or preventing the onset of POAG in individuals with elevated TOP.

The trabecular meshwork (TM) consists of the elements: the uveal and corneoscleral meshworks and the juxtacanalicular connective tissue (JCT). The TM, together with the endothelial lining of Schlemm's canal, the collector channels and the episcleral venous system, comprise the conventional aqueous outflow pathway, which accounts for the majority of total aqueous humor drainage. Thus, the TM plays an essential role in regulating TOP, with the JCT and Schlemm's canal endothelium (SCE) generally believed to be the major site of resistance to aqueous outflow. Cells in the uveal and corneoscleral meshwork portions of the TM may also have important roles in regulation of aqueous outflow, such as phagocytosis of debris and foreign bodies, modulating permeability of SCE and extracellular matrix (ECM) production.

In glaucomatous eyes, TM displays several pathologic features in addition to elevated flow resistance. Firstly, TM cellularity reduction is observed in glaucomatous eyes compared to age-matched control eyes. This likely leads to adhesion of trabecular lamellae, thickening of trabecular beams and accumulation of fibrillary plaque material, all of which disturb TM microstructure. Secondly, the mechanical properties of the TM itself are altered: compared to healthy TM, glaucomatous TM has increased stiffness that may be associated with increased outflow resistance.

### STATEMENT REGARDING FEDERAL FUNDING

This invention was made with government support under Grant Nos. EY008098 and EY025643 awarded by the National Institutes of Health. The government has certain rights in the invention.

### SUMMARY

In light of the difficulties and costs associated with use of autologous TM cells, TM stem cells, or stem cells produced from other sources, it has been found that adipose-derived stem cells (ADSCs) can easily be differentiated into TM cells, either by culturing the ADSCs with primary trabecular meshwork cells, extracellular matrix produced by trabecular meshwork cells, and/or trabecular meshwork cell-conditioned medium, e.g., in the eye of a patient. Therefore a method of preparing functional trabecular meshwork cells from adipose-derived stem cells is provided along with functional trabecular meshwork cells prepared from adipose-derived stem cells.

In one aspect, a method of repairing or regenerating the aqueous outflow pathway of an eye of a patient is provided. The method comprises introducing, e.g. injecting or otherwise placing, into an eye of the patient, e.g. the TM of the patient, an adipose-derived stem cell (ADSC) or a functional TM cell prepared from an ADSC. In another aspect, a method of implanting trabecular meshwork (TM) cells in an eye of a patient, e.g. the TM of a patient, in need thereof is provided. The method comprises introducing, e.g. injecting or otherwise placing, into the eye of the patient an adipose-derived stem cell (ADSC) or a functional TM cell prepared from an ADSC. In another aspect, a method of treating glaucoma in a patient in need thereof is provided. The method comprises introducing, e.g. injecting or otherwise placing, into an eye of the patient an ADSC or a functional trabecular meshwork (TM) cell prepared by differentiation of an adipose-derived stem cell (ADSC). In all cases, the ADSCs delivered or used to produce the functional TM cells are autologous, that is they are the patient's own ADSCs obtained from the patient's adipose tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D provide data showing expression of TM cell markers in cells cultured as described in the Example.

FIGS. 2A-2B provide data showing phagocytic function of cells as described in Example.

FIGS. 3A-3D provide data showing organization of actin networks, and MYOC expression in cells treated as described in the Example.

FIGS. 4A-4F provide data relating to the injection of functional TM cells into the eyes of mice as described in the Example.

FIGS. 5A-5F provide data relating to intraocular pressure and outflow in mouse eyes treated as described in the Example. FIGS. 5B-5F graph flow rate (microliters (μL) per minute) vs intraocular pressure (mmHg).

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

As used herein, the terms “comprising,” “comprise” or “comprised,” and variations thereof, are meant to be open ended. The terms “a” and “an” are intended to refer to one or more.

As used herein, the term “patient” or “subject” refers to members of the animal kingdom including but not limited to human beings.

Provided herein are methods of making TM cells from ADSCs, and methods of using the cells, for reconstituting trabecular meshwork in a patient, for example for treating glaucoma in the patient, and for repairing or regenerating the aqueous outflow pathway in the eye of a patient.

Although bone marrow-derived (BM-MSCs) and ADSCs share a lot of characteristics, ADSCs are easier and less invasive to obtain from patients, and may have other advantages.

TABLE 1
Usefulness of various stem cell sources for obtaining TM cells.

| | TMSCs | ADSCs | BM-MSCs | iPSCs |
|---|---|---|---|---|
| Autologous | No | Yes | Yes | Yes |
| Allogeneic | Yes | Yes | Yes | Yes |
| Induction | One simple | One simple | One simple | Multiple |
| process | step | step | step | steps |
| Contamination | Low | Low | high | Highest |
| risk | | | | |
| Virus concern | No | No | No | Yes |
| Risk | low | Minimal | high | highest |

Multipotent mesenchymal stem cells can be readily isolated from bone marrow or adipose tissue. ADSCs can be harvested using minimal invasive procedures with little risk and discomfort compared to bone marrow-derived stem cells. So ADSCs are a great autologous resource. Autologous ADSCs can be retrieved from either liposuction aspirates or subcutaneous adipose tissue fragments and are easily expanded in vitro. For non-high risk patients, stocked frozen ADSCs can be delivered and thawed for allogeneic transplantation. Expanded ADSCs can be induced to differentiate into TM cells and transplanted into patient anterior chamber where they can home to the TM tissue and reconstruct the outflow pathway.

ADSCs are induced by culturing on a special extracellular matrix generated from either decellularized TM tissue or cultured TM cells (e.g., decellularized trabecular meshwork (e.g., TM-ECM) obtained by decellularization or devitalization of trabecular meshwork tissue obtained from an animal, e.g., a mammal, or from a devitalized cell culture of either primary trabecular meshwork cells, or trabecular cells prepared from ADSCs as described herein), and/or in a TM cell-conditioned medium (e.g., medium collected from a cell culture of either primary trabecular meshwork cells, or trabecular cells prepared from ADSCs as described herein). In one aspect, ammonium hydroxide is used to decellularize the TM tissue. In another aspect, the ADSCs are cultured on TM-ECM with TM cell-conditioned medium. Induced ADSCs express TM cell markers, such as CHI3L1, AQP1 and are phagocytic—having the ability to ingest inactivated S. aureus bioparticles. Further, the induced ADSCs are responsive to dexamethasone treatment expressing MYOC, a glaucoma associated gene, which is a specific characteristic of TM cells. TM stem cells (TMSCs) were used for the same purpose (Du Y, et al., Multipotent stem cells from trabecular meshwork become phagocytic TM cells. Investigative ophthalmology & visual science 2012; 53:1566-1575 and Du Y, et al., Stem cells from trabecular meshwork home to TM tissue in vivo. Investigative ophthalmology & visual science 2013; 54:1450-1459). Although promising progress has been made, obtaining autologous TM cells requires surgery on the eye. We have previously successfully induced ADSCs into corneal stromal keratocytes (Du Y, et al., Adipose-derived stem cells differentiate to keratocytes in vitro. Molecular vision 2010; 16:2680-2689).

As indicated herein, the readily-available ADSCs are useful for preparation of functional TM cells. Because the ADSCs are used to prepare cells for injection in a patient's eye, the ADSCs are preferably autologous, but are readily-obtained from a patient's fat deposits, such as, e.g., from above the patient's superior iliac spine. allogeneic, or even xenogeneic ADSCs may be used with consideration of management of immune rejection. Adipose-derived stem cells ADSCs, can be prepared by any useful method, including that shown below. U.S. Pat. Nos. 6,777,231 and 7,470,537, Bunnell et al., Adipose-derived Stem Cells: Isolation, Expansion and Differentiation, Methods. 2008 June; 45(2): 115-120. doi:10.1016/j.ymeth.2008.03.006 and Mizuno et al., Concise Review: Adipose-Derived Stem Cells as a Novel Tool for Future Regenerative Medicine, Stem Cells 2012; 30:804-810, each of which is incorporated herein by reference in its entirety, describe adipose-derived stem cells and methods of making adipose-derived stem cells.

As used herein, a “progenitor cell” is a cell type in a cell lineage that can differentiate into another cell type in that lineage. ADSCs are in their broadest sense, cells obtained from adipose tissue and having the ability to differentiate to functional TM cells as described herein. Additional markers of ADSCs include, without limitation: presence of nestin (e.g., HGNC: 7756; Entrez Gene: 10763; Ensembl: ENSG00000132688; OMIM: 600915; UniProtKB: P48681), the presence of OCT4 (e.g., a product of the POU5F1 gene, HGNC: 9221 Entrez Gene: 5460 Ensembl: ENSG00000204531 OMIM: 164177 UniProtKB: Q01860), and the ADSCs have the ability to differentiate into functional TM cells e.g., in (1) co-culture with TM cells; (2) culture on TM-secreted extracellular matrix in TM-conditioned medium; (3) culture on TM-secreted ECM and cell culture medium, e.g., as described in the example below; and (4) by introduction of ADSCs, e.g. autologous ADSCs, into the anterior chamber and/or the trabecular meshwork of a patient's eye and subsequent differentiation of the cells into functional TMs within the trabecular meshwork. As used herein, a “marker” is a detectable physical or functional characteristic or phenotype of a cell, such as, without limitation: an expressed protein or nucleic acid, a non-secreted or secreted composition, a physical quality of the cell, an activity of the cell, or a function of the cell.

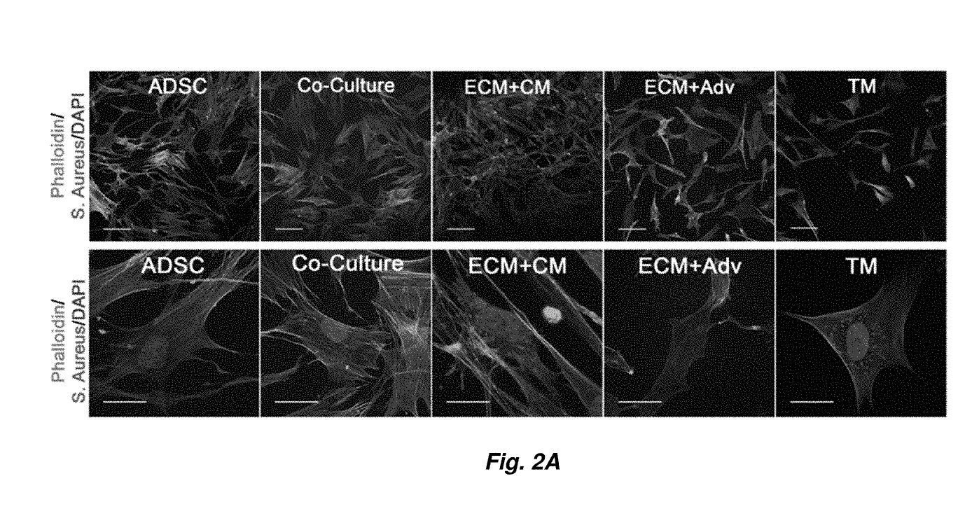

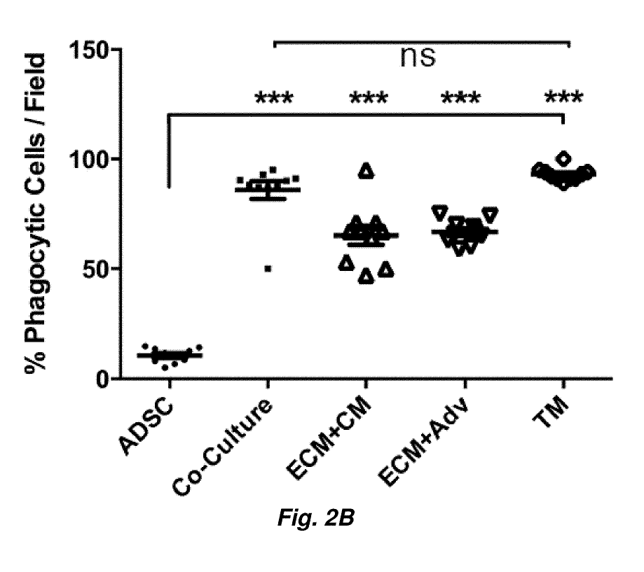

As used herein, a “functional TM cell” is a cell differentiated from ADSCs, or from other progenitor cells, and having certain markers, including functionality, of normal TM cells. As shown below, functional TM cells prepared according to methods described herein expressed TM cell markers (FIG. 2), they were phagocytic (FIG. 2), they could home to the TM tissue in vivo and integrated into the TM tissue expressing TM marker AQP1 (FIG. 6). In one aspect of the described method, the functional TM cells are produced by differentiation of progenitor cells, e.g., ADSCs, which are capable of differentiating into the functional TM cells (1) by co-culture with TM cells; (2) by culture on TM-secreted extracellular matrix in TM-conditioned medium; (3) by culture on TM-secreted ECM and cell culture medium; by introduction of ADSCs, e.g. autologous ADSCs, into the anterior chamber and/or the trabecular meshwork of a patient's eye and subsequent differentiation of the cells into functional TMs within the trabecular meshwork. Characteristics of functional TM cells include the following markers: CHI3L1 (chitinase-3-like protein 1, e.g., MIM (OMIM, Online Mendelian Inheritance in Man®):601525, (Entrez) Gene ID: 1116); AQP1 (Aquaporin 1, e.g., MIM:107776, Gene ID: 358); MGP (Matrix Gla Protein, e.g., MIM: 154870 and Gene ID: 4256); MYOC (myocilin, trabecular meshwork inducible glucocorticoid response, e.g., MIM:601652, Gene ID: 4653) and ANGPTL7 (angiopoietin-like 7, e.g., HGNC:HGNC:24078 and Gene ID: 10218) after induction and after dexamethasone stimulation; and/or are phagocytic with the ability to ingest inactivated S. aureus bioparticles, and when transferred into an eye, the cells integrate into the TM, maintain normal IOP, and outflow pathway homeostasis within normal range of outflow facility, and in one aspect having more than one of the preceding markers in any combination, and in another aspect, having all of the preceding markers. The stem cells can be any stem cell able to differentiate into functional keratocytes that produce collagen, keratan sulfate and keratocan. Examples of suitable stem cells are corneal stromal stem cells and adipose-derived stem cells, and in one embodiment, human corneal stromal stem cells and human adipose-derived stem cells. In one preferred embodiment, the stem cells are human corneal stromal stem cells.

According to one aspect of the invention, provided herein is a method of producing functional trabecular meshwork (TM) cells. The method comprises culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to TM cells. The time sufficient to cause differentiation of the ADSCs to TM cells is typically at least 2 days, e.g., from two to 14 days, including any increment therebetween, or from five to seven days, including any increment therebetween. The ADSCs are cultured ex vivo, that is, in a suitable vessel, and under suitable conditions for maintenance, growth and/or expansion of the population of cells. Cells are cultured in any suitable cell culture medium—a vast number of which are known to those of ordinary skill in the art and which are commercially available. Eukaryotic cells are often cultured in medium containing animal serum, such as bovine calf serum. In use, for differentiation of cells, such as autologous cells, for use in a patient's eyes, introduction of xenogeneic or allogeneic, e.g. non-self, proteins and peptides, is undesirable. As such, serum-free media is typically preferred for growth of ADSCs and for differentiation of the ADSCs to functional TM cells. The media used in the Examples below is merely exemplary, and one of ordinary skill in the art can readily determine if a different medium would be effective for the stated purpose. As with the options for cell culture medium, options for culture vessels are many. Where the cells are cultured in the presence of TM cells, the TM feeder cells is optionally physically separated from the ADSCs by a suitable membrane or porous material, permitting passage of growth factors or other constituents useful for differentiating the ADSCs to functional TM cells, but not permitting contamination of the ADSCs or functional TM cells prepared from the ADSCs with the non-autologous cells used as feeder cells. Cells can be plated, or grown in a suitable bioreactor in suitable medium or conditioned medium. For example TM ECM can be deposited on beads or other surfaces and used to grow and differentiate the ADSCs. In one aspect, TM cells are grown on a substrate, such as a plate, flask, tube, bead, particle, etc., and then are decellularized to produce a surface coated with the TM ECM, which will support growth and differentiation of ADSCs, such as autologous ADSCs (to a patient). As an be appreciated by one of ordinary skill, there are many culture methods and options that may be used to produce functional TMs as described herein.

In another aspect of the invention, the TM is populated in vivo with functional TM cells by introduction of ADSCs, for example in one aspect, autologous (the patient's own) ADSCs, into the trabecular meshwork of the patient, e.g. by direct injection into the anterior chamber of the eye, where the ADSCs differentiate in the TM in situ into functional TM cells. Thus, according to one aspect, adipose tissue is acquired from a patient to be treated with functional TM cells. ADSCs are obtained from the patient's adipose tissue according to any acceptable protocol. The ADSCs are then introduced into the patient's TM, for example by direct injection into the anterior chamber of the patient's eye in need of generation, regeneration or repair of its TM. The ADSCs will differentiate into functional TM cells at the TM of the patient. In one aspect, the patient has glaucoma in the treated eye.

For use of TM ECM to prepare the functional TM cells, a culture of TM cells, such as a confluent culture of TM cells may be utilized to produce the TM ECM, which is prepared by decellularization of the confluent culture of TM cells. A number of decellularization methods are know in the art and may be useful for preparation of decellularized TM ECM, but in one aspect ammonium hydroxide, e.g., 0.02N ammonium hydroxide, is used to decellularize the TM cells to produce TM ECM. Other compositions can be used to decellularize the TM ECM, such as by treatment with enzymes such as trypisin, pepsin, papain, and/or DNAse, surfactants, detergents, emulsifiers, solvents (e.g. ethanol), and osmotic stress, or combinations thereof, including any necessary washing sand disinfecting steps. The TM cells used to prepare the ECM material can be of any source, such as from allogeneic sources, e.g. from a human organ donor, or from xenogeneic sources, such as from, e.g., bovine or porcine TM tissue obtained from an abbatoir, and even by use of previously-differentiated functional TM cells prepared from a method described herein.

Conditioned medium is prepared by culturing cells, and in the context of the present invention, TM cells or functional TM cells, for a time period sufficient for the cells to produce necessary factors, such as cytokines, growth factors, etc., such that the conditioned media is useful in differentiating ADSCs to functional TM cells. The time period typically ranges from 12 hours to one week, including any and all increments therebetween. The conditioned medium is aspirated, or is otherwise removed from the TM cells or functional TM cells, and is then used in culture, alone or in combination with additional medium, such as fresh medium, to differentiate the ADSCs to functional TM cells. In one aspect, when functional TM cells are grown to a sufficient density, the medium is removed and is saved for future use as conditioned medium, and optionally a portion of the functional TM cells are saved, e.g. frozen at −80° C. for future use either in the patient, or to support growth of future cultures of ADSCs to be differentiated to functional TM cells.

In one aspect, a method of treating glaucoma in a patient is provided. In another aspect, a method of implanting trabecular meshwork (TM) cells in an eye of a patient is provided. In a further aspect, a method of repairing or regenerating the aqueous outflow pathway of an eye of a patient is provided. In such a method, adipose tissue is obtained from the patient as a source of ADSCs, and an enriched population of ADSCs are prepared from the adipose tissue. The patient's ADSCs are induced to differentiate to TM cells as described herein. In one aspect, the patient's ADSCs are introduced into the TM of an eye of a patient, e.g., by direct injection into the anterior chamber of the eye, and differentiate to functional TM cells at or within the existing TM of the patient. In another aspect, the patient's ADSCs are co-cultured with TM cells. In another aspect, the patient's ADSCs are cultured on ECM obtained from decellularization of a culture of TM cells, e.g. by decellularizing a xenogeneic or allogeneic, confluent or sub-confluent culture of TM cells with 0.02N Ammonium hydroxide, as described below, or by any useful method. In another aspect, the ADSCs are induced to differentiate to TM cells by culture in medium conditioned with TM cells. In another aspect, the ADSCs are induced to differentiate to TM cells by culture on ECM obtained from decellularization of a culture of TM cells in medium conditioned with TM cells. In a further aspect, a functional TM cell is provided that is prepared from ADSCs by any method described herein.

### Example—Human Adipose-Derived Stem Cells Differentiate into Phagocytic Trabecular Meshwork Cells and Integrate into Mouse TM Tissue

The trabecular meshwork (TM) is an ocular tissue that maintains intraocular pressure (TOP) within a safe range. Glaucoma patients have reduced TM cellularity and, frequently, elevated IOP. However, no current treatments for glaucoma directly target TM cell loss to restore function and normalize IOP. To establish a stem cell approach to restoring TM cellularity and function, human adipose-derived stem cells (ADSCs) were induced to differentiate to TM cells. These ADSC-TM cells displayed a TM cell-like genotypic profile, became phagocytic, and responded to dexamethasone stimulation by expressing myocilin (MYOC) and forming cross-linked actin networks (CLANs). After transplantation into normal mouse eyes, they integrated into the TM, expressed TM cell markers, maintained normal IOP and outflow pathway homeostasis within normal range of outflow facility. Cell migration and affinity experiments indicated that the chemokine pair CXCR4/SDF1 may play an important role in ADSC-TM cell homing and integration. Our study demonstrates the possibility of applying autologous or allogeneic ADSC-TM cells in vivo and provides a regenerative strategy to restore the structure and function of TM tissue in glaucomatous eyes.

Current pharmacological and surgical therapies seek to lower IOP by facilitating aqueous humor outflow and suppressing aqueous humor production; however, these strategies fail to directly target TM cellularity loss, which is likely an important pathophysiologic cause of glaucoma. Theoretically, repopulation of the TM by stem cells could compensate for decreased cellularity in glaucomatous eyes and restore TM function, thus reducing IOP. This idea is indirectly supported by studies of human eyes receiving laser trabeculoplasty, in which TM cells were diffusely stimulated by laser resulting in increased cell division and migration of cells within the TM to repopulate the burned site. The possible mechanism behind the effect of TM cell restoration might be either through direct interaction with the extracellular matrix of TM or through a paracrine effect on the remaining endogenous cells. This inspires application of stem cells for tissue regeneration for POAG patients.

It has been reported that there are tissue-specific stem cells in the TM tissue. Human TM stem cells have been successfully isolated and characterized. When intracamerally injected in the mouse anterior chamber, these stem cells can home to TM tissue and maintain mouse IOP in the normal range (Du Y, et al. Stem cells from trabecular meshwork home to TM tissue in vivo. Invest Ophthalmol Vis Sci. 2013; 54(2):1450-9). When injected into the anterior chamber of mice with laser damage to the TM, the stem cells can integrate into damaged TM tissue and rescue outflow facility.

As alternatives to harvesting TM stem cells, which are likely depleted/absent in glaucoma patients, other stem cell types have been explored for TM regeneration such as mesenchymal stem cells (MSCs) (Manuguerra-Gagne R, et al. Transplantation of mesenchymal stem cells promotes tissue regeneration in a glaucoma model through laser-induced paracrine factor secretion and progenitor cell recruitment. Stem Cells. 2013; 31(6):1136-48) and Induced pluripotent stem cells (iPSCs)(Abu-Hassan D W, et al. Induced pluripotent stem cells restore function in a human cell loss model of open-angle glaucoma. Stem Cells. 2015; 33(3):751-61; Ding Q J, et al. Induction of trabecular meshwork cells from induced pluripotent stem cells. Invest Ophthalmol Vis Sci. 2014; 55(11):7065-72; and Zhu W, et al. Transplantation of iPSC-derived TM cells rescues glaucoma phenotypes in vivo. Proc Natl Acad Sci USA. 2016; 113(25):E3492-500). After bone marrow-derived MSCs injected into rat anterior chamber, IOP was rapidly reduced to normal and TM structure was restored at one month (Manuguerra-Gagne R, et al. Stem Cells. 2013; 31(6):1136-48). iPSCs are reprogrammed cells with characteristics similar to embryonic stem cells. They can be derived from patients' own dermal fibroblasts or blood samples or urine samples. Both MSCs and iPSCs are good candidates as cell therapy resources.

In addition to MSCs and iPSCs, another candidate is human adipose-derived stem cells (ADSCs). They can be obtained in large quantities with minimally invasive procedures (Frese L, et al. Adipose Tissue-Derived Stem Cells in Regenerative Medicine. Transfus Med Hemother. 2016; 43(4):268-74). We have successfully induced human ADSCs to differentiate into corneal keratocytes (Du Y, et al. Adipose-derived stem cells differentiate to keratocytes in vitro. Mol Vis. 2010; 16:2680-9), a cell type derived from neural crest and thus sharing the same embryonic origin as TM cells. That study gave us a hint that ADSCs might be able to differentiate into TM cells. Here, we demonstrate that ADSCs can be induced to differentiate into TM-like cells by examining their gene expression, their response to dexamethasone (DEX) exposure, and their functions such as phagocytosis and ability to maintain aqueous humor dynamics in vivo. We additionally compared the specific integration of primary ADSCs and ADSC-TM grafts into mouse TM tissue and explored possible mechanism of stem cell homing. These studies provide the potential for a regenerative therapeutic strategy to restore functions of glaucomatous TM tissue and thus protect the eye from vision loss.

### Materials

Primers used in qPCR are shown in Table 2. Primary antibodies used are shown in Table 2. For immunofluorescent staining, anti-mouse Alexa-488, anti-rabbit Alexa-488, anti-rabbit Alexa-647, nuclear dye DAPI, and Vybrant DiO were obtained from Invitrogen Life Technologies (ThermoFisher Scientific, Pittsburgh, Pa.).

TABLE 2
Primary antibodies used for immunostaining and Western blotting.

| Antibody | Type | Source | Catalog# |
|---|---|---|---|
| CHI3L1 | Goat polyclonal | R&D | AF2599 |
| MGP | Mouse monoclonal | Santa Cruz | Sc-81546 |
| AQP1 | Rabbit polyclonal | Santa Cruz | Sc-20810 |
| Myocilin | Rabbit polyclonal | Santa Cruz | Sc-20976 |
| OCT4 | Rabbit polyclonal | Santa Cruz | Sc-9081 |
| Nestin | Mouse monoclonal | EMD Millipore | MAB5326 |
| β-actin | Mouse monoclonal | Biolegend | 643802 |

### Preparation of Human ADSC Cells and TM Cells

Human TM cells were isolated and cultured as previously described (27). In brief, deidentified human corneas were obtained from the Center for Organ Recovery and Education (Pittsburgh, Pa.). Eyes were preserved within 12 hours post mortem and stored no more than 5 days before harvesting for cell culture. Cells from three donors at 46, 58 and 62 years of age were used in the experiments shown. Human ADSCs were obtained from three individuals at 34, 36 and 38 years old undergoing elective lipoaspiration. ADSCs were isolated by collagenase digestion and differential centrifugation as previously described (Du Y, et al. Adipose-derived stem cells differentiate to keratocytes in vitro. Mol Vis. 2010; 16:2680-9 and Aksu A E, et al. Role of gender and anatomical region on induction of osteogenic differentiation of human adipose-derived stem cells. Ann Plast Surg. 2008; 60(3):306-22).

### Differentiation of ADSC-TM Cells

Three methods were used and compared to generate ADSC-TM cells. (1) Co-culture: A 3D co-culture environment was established by using cell culture inserts (0.4 μm pores; Corning Inc, Corning, N.Y.) placed in 6-well plates. TM cells were seeded into the insert (upper chamber) without contacting the ADSCs on the bottom of the culture wells. (2) ECM+CM: Extracellular matrix (ECM) generated from TM cells was prepared by lysing completely confluent TM cells using 0.02N ammonium hydroxide. TM-conditioned medium (CM) was collected 3 days after passaging TM cells at p3-p5 for about 80% confluence and centrifuged at 10,000 RPM for 30 min to remove possible remaining cells in the medium and stored at 4° C. for future use within 1 month. ADSCs were then cultured on TM-secreted ECM in media composed of 50% DMEM/F12-10% FBS and 50% CM. (3) ECM+Adv: ECM from TM cells was prepared as described above. ADSCs were cultured on the ECM in advanced MEM (Adv, ThermoFisher) with 0.1M ascorbic acid-2-phosphate (Sigma-Aldrich, St. Louis, Mo.) without serum. Adv MEM has been used for differentiation of neural crest derived keratocytes. Culture media were changed every 3 days and induction efficiency in the above three approaches was assayed for up to 14 days.

### Phagocytosis Assay

pHrodo Red S. aureus Bioparticles conjugate (ThermoFisher) was used in our experiments. ADSC-TM cells, primary ADSCs and TM cells at the same passage number were cultured on coverslips in 6-well plates until they were 70% confluent. S. aureus bioparticles were diluted by DMEM/F12 to make a 1 mg/ml dispersed suspension and incubated with each type of cell for 4 hours at 37° C., avoiding light. Cell were then gently washed with 1×PBS, trypsinized, centrifuged at 1200 rpm at 4° C. for 5 min, resuspended in DMEM/F12, and reseeded onto coverslips in 6-well plates. After incubation at 37° C. for 3 hours, cells were fixed in 4% paraformaldehyde and stained with 4,6-diamidino-2-phenylindole (DAPI; ThermoFisher) at 1 μg/mL and phalloidin conjugated with Alex-633 (ThermoFisher) at 1:500 for 1 hour. Using a confocal microscope (Olympus FluoView FV1000), the internalization of S. aureus bioparticles was observed and imaged, and the quantification of phagocytosis was established by calculating the proportion of bioparticle-ingesting cells as a fraction of the total number of cells in randomly selected fields (n=10).

### DEXamethasone Stimulation of CLAN Formation

Human ADSCs were differentiated for 10 days and exposed to 200 nM DEX for 7 days. Differentiated cells without DEX but cultured in the same medium containing DMSO as DEX dissolved in were used as controls. Cells were stained with Phalloidin-633 and DAPI. Images were taken on a confocal microscope for at least 10 individual fields of 40× oil view per condition. The morphology of putative CLANs was examined in detail at higher magnification. CLANs are three-dimensional, geodesic-dome-like structures of cellular microfilaments. In two dimensional microscopic views, CLANs appear to be web-like structures with numerous hubs and spokes (45, 59). The number of CLANs-forming cells and non-CLAN containing cells in a field were counted and the percentage of cells that developed CLANs was determined and compared among groups. At least 10 fields of each condition were counted and averaged. CLAN counting was done in a masked manner.

Transplantation of ADSC Cells into the Anterior Chamber of Mice

All mice were anesthetized by intraperitoneal injection of ketamine hydrochloride (50 mg/kg) and xylazine (5 mg/kg) (IVX Animal Health, Inc., St. Joseph, Mo.) in Dulbecco's PBS. The eyes were washed with PBS and anesthetized by topical drops of proparacaine HCl (Falcon Pharmaceuticals, Fort Worth, Tex.). Intracameral injection following the procedures previously described (31). In brief, a corneal tunnel was made using a 30-gauge needle. An air bubble was then introduced into the anterior chamber by injecting a 1.5 μl volume of air with a microsyringe (Hamilton, Reno, Nev.) fitted with a 33-gauge beveled needle (Hamilton). Next, 10,000 cells (ADSC-TMs, primary ADSCs, fibroblasts, n=25 in each group) in 2 μL DMEM/F12 were injected into the anterior chamber with a microsyringe fitted with a 33-gauge blunt needle (Hamilton). An equal volume of DMEM/F12 was injected as sham control. After the injection, one drop of Goniovisc (2.5% hypromellose Ophthalmic solution, Hub Pharmaceuticals, Rancho Cucamonga, Calif.) was applied to protect the ocular surface from drying during anesthesia.

### Intraocular Pressure Measurement

IOP was measured using a rebound tonometer for rodents (TonoLab; Colonial Medical Supply, Franconia, N.H.) before and after cell injection at 3, 5, 7, 10, 14, 21 and 30 days. IOP was consistently measured between 3:00 pm to 5:00 pm using the same anesthesia regimen as described above. IOP data were collected in a masked manner for all mice.

### Measurement of Aqueous Humor Outflow Facility

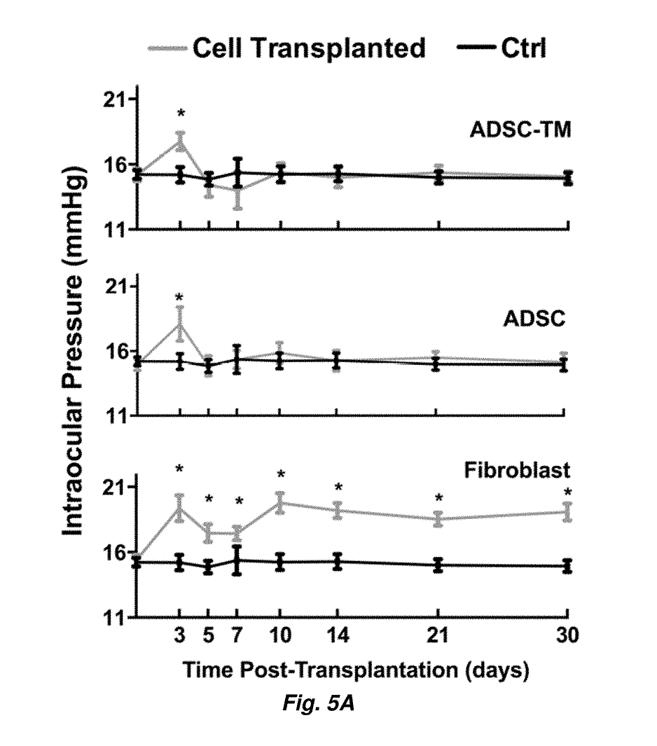

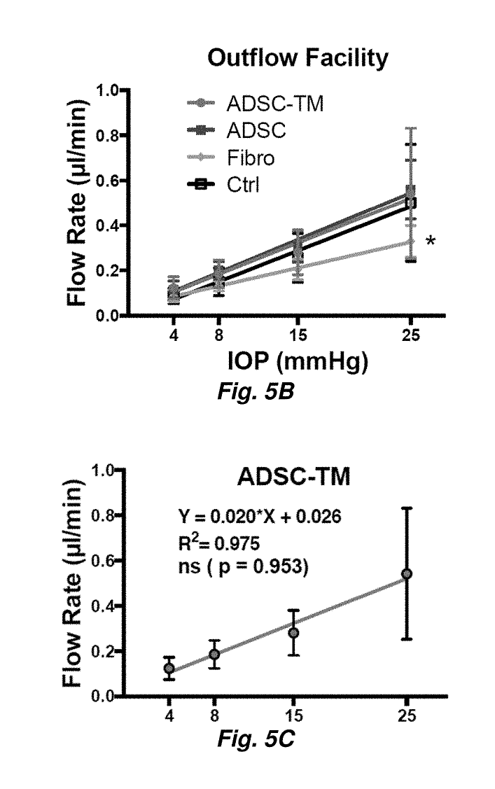

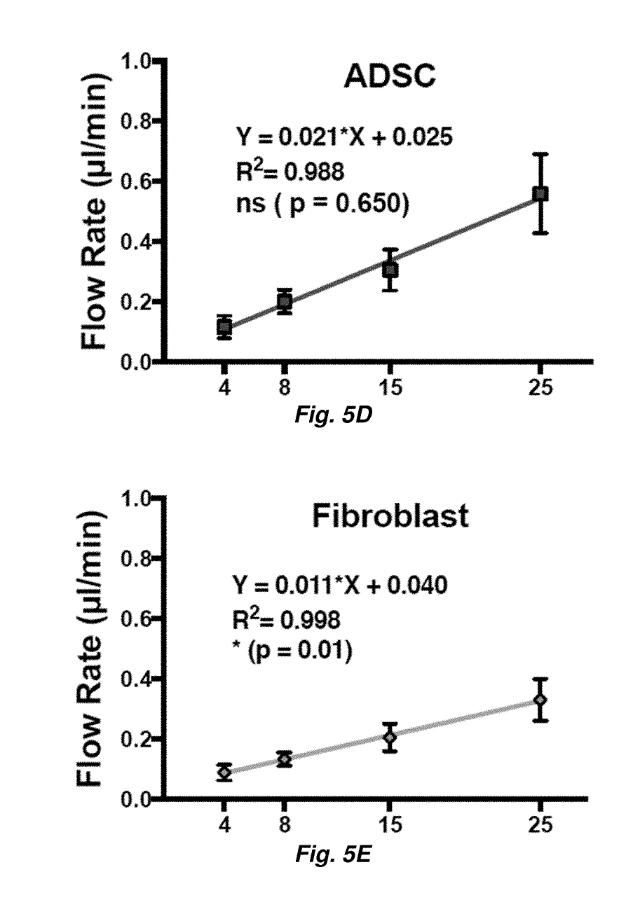

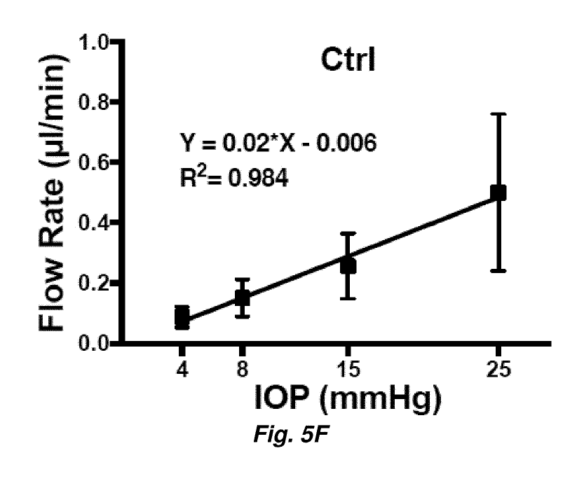

Mice were anesthetized to measure IOP and sacrificed at day 30 post injection and eyes were enucleated. Outflow facility measurement followed the procedures described by Lei et al (51) with minor modifications. The perfusion system consisted of a computer-controlled syringe pump (Harvard Apparatus, Hilliston, Mass.) that delivered a variable flow rate (Q) to the anterior chamber so as to maintain a desired IOP, as monitored by a pressure transducer (Honeywell, Ft. Washington, Pa.) connected to a computer control system (Labview software; National Instruments, Austin, Tex.). The anterior chamber was cannulated with a microinjection glass pipette connected to the pressure transducer. A 25 μl Hamilton syringe filled with PBS was loaded onto the syringe pump. Eyes were perfused with PBS at constant pressures of 4 mmHg, 8 mmHg, 15 mmHg and 25 mmHg sequentially for at least 15 min at each pressure level (FIGS. 5B-5F). The average flow rate at each set pressure was calculated. We used the Goldmann equation as described by Lei et al (51, 60): F=(Po−Pv)C+Fu, in which Po is the IOP (mmHg), F is the rate of aqueous formation (equivalent to pump flow rate at steady state in enucleated eyes), C is the conventional outflow facility, and Pv is the episcleral venous pressure (equal to zero in enucleated eyes), Fu is the pressure-independent (unconventional) outflow rate. Linear regression was used to fit the pressure-flow data and hence estimate outflow facility: C=F/Po−Fu. In FIGS. 5B-5F, facility is the slope of the regression line. Data from a given eye were only accepted when γ^{̂2}>0.95.

Quantitative Reverse Transcription—Polymerase Chain Reaction (qPCR)

Cells were lysed with RLT buffer (RNeasy mini kit; Qiagen, Valencia, Calif.) and RNA was isolated following the manufacturer's instructions, including treatment with DNase I (Invitrogen) and concentration by ethanol precipitation. cDNAs were transcribed from the RNAs using XLAScript™ cDNA MasterMix (WorldWide Medical Products Inc, Bristol, Pa.). qPCR was performed by direct dye binding (SYBR Green; Applied Biosystems) as previously described (31). Primers were designed using online software (Primer3; http://bioinfo.ut.ee/primer3-0.4.0/primer3/), with the sequences shown in Table 3. Amplification of 18S rRNA was performed for each cDNA (in triplicate) for normalization of RNA content. Relative mRNA abundance was calculated as the Ct for amplification of a gene-specific cDNA minus the average Ct for 18S expressed as a power of 2 (2^{-ΔΔCt}). Three individual gene-specific values thus calculated were averaged to obtain mean SD.

TABLE 3
Primer sequences used in quantitative RT-PCR

| Gene Name | |
|---|---|
| (Genbank | |
| Accession No) | DNA Sequence |
| 18S Ribosomal | Forward: CCCTGTAATTGGAATGAGTCCAC |
| RNA | (SEQ ID NO: 1) |
| (NR_003286.2) | Reverse: GCTGGAATTACCGCGGCT |
| | (SEQ ID NO: 2) |
| CHI3L1 | Forward: GATGTGACGCTCTACGGCAT |
| (NM_001276.2) | (SEQ ID NO: 3) |
| | Reverse: TGATGAAAGTCCGGCGACTC |
| | (SEQ ID NO: 4) |
| MGP | Forward: GCCGCCTTAGCGGTAGTAAC |
| (NM_000900.4) | (SEQ ID NO: 5) |
| | Reverse: TCTCTGCTGAGGGGATATGA |
| | (SEQ ID NO: 6) |
| AQP1 | Forward: CTGCACAGGCTTGCTGTATG |
| (NM_198098.3) | (SEQ ID NO: 7) |
| | Reverse: TGTTCCTTGGGCTGCAACTA |
| | (SEQ ID NO: 8) |
| Myocilin | Forward: AAGCCCACCTACCCCTACAC |
| (NM_000261.1) | (SEQ ID NO: 9) |
| | Reverse: TCCAGTGGCCTAGGCAGTAT |
| | (SEQ ID NO: 10) |
| OCT4 | Forward: GTGGAGGAAGCTGACAACAA |
| (NM_002701.4) | (SEQ ID NO: 11) |
| | Reverse: GGTTCTCGATACTGGTTCGC |
| | (SEQ ID NO: 12) |
| Nestin | Forward: AAGATGTCCCTCAGCCTGG |
| (NM_006617.1) | (SEQ ID NO: 13) |
| | Reverse: GAGGGAAGTCTTGGAGCCAC |
| | (SEQ ID NO: 14) |

### Western Blot

Cells lysates were collected using RIPA buffer (Santa Cruz Biotechnology, Santa Cruz, Calif.). heated at 95° C. for 5 minutes, and sonicated until solubilized. β-mercaptoethanol was added to the lysates to a final concentration of 1% and the mixture was heated at 70° C. for 20 minutes. Samples were mixed with 2× Laemmli loading buffer (BIO-RAD, Hercules, Calif.) loaded to 8-16% Tris-Glycine Gel (ThermoFisher) and electrophoresis was performed for 1 hour at 200 V. Protein was transferred to a polyvinylidene difluoride membrane (Millipore) and blocked for 1 hour at RT in Odyssey blocking buffer (LI-COR Biotechnology, Lincoln, Nebr.). Membranes were incubated with primary antibodies diluted in blocking buffer with 0.01% Tween-20 followed by incubation with goat anti-mouse, goat anti-rabbit, or donkey anti-goat secondary antibodies (IRDye 680LT, IRDye 800CW, and IRDye 800CW, respectively). The fluorescent signal was captured on an infrared imager (Odyssey Infrared Imager; LI-COR Biosciences, Lincoln, Nebr.).

### Immunohistochemistry

Cells cultured directly on 35-mm tissue-culture dishes or NUNC Lab-Tek 8-chamber slides (ThermoFisher) were rinsed briefly in PBS, fixed in 4% paraformaldehyde at RT for 15 minutes, rinsed in PBS, and stored at 4° C. in 50% glycerol and 50% PBS (v/v) until staining. Enucleated mouse eyes were rinsed and fixed in 1% paraformaldehyde and embedded in optimal cutting temperature (OCT) embedding compound (Tissue-Tek OCT; Electron Microscopy Sciences, Hatfield, Pa.), cut into 8 μm sections, and stored at −20° C. until staining. Nonspecific binding was blocked with 1% bovine serum albumin (ThermoFisher). Sections were incubated overnight at 4° C. with primary antibodies (shown in Table 1). After three washes, anti-mouse Alexa 488 or 546, anti-rabbit Alexa 546 or 647, or anti-goat Alexa 546 secondary antibodies, and nuclear dye DAPI were added and incubated for 2 hours at RT. Samples were imaged using a confocal microscope (Olympus) with a 40× or 60× oil objective. Microscopic analysis was carried out with the FluoView software.

### Assessment of Apoptosis by TUNEL Staining

The TUNEL assay was performed using a cell death detection kit (In Situ Cell Death Detection Kit, TMR red; Roche Molecular Biochemicals) following the manufacturer's protocol on cryopreserved tissue. Nuclei were stained with DAPI. At least three independent eyes from each condition and at least nine cryosections of each condition were stained, imaged and counted using a confocal microscope.

### Statistical Evaluation

At least three biological independent experiments were performed for both in vitro and in vivo data. All statistical analyses were performed with one-way ANOVA followed by Tukey's post-test or Dunnett's post-test, or two-way ANOVA followed by Sidak's post-test. Values were considered statistically significant if p was less than 0.05.

### Transmission Electron Microscopy (TEM)

The ultrastructure of mouse TM was examined by TEM, following previously described methods (Yun H, et al. A laser-induced mouse model with long-term intraocular pressure elevation. PLoS One. 2014; 9(9):e107446). In brief, mouse eyeballs were fixed in cold 2.5% glutaraldehyde (EM grade, Taab Chemical) in PBS and post-fixed in aqueous 1% osmium tetroxide (Electron Microscopy Sciences, Hatfield, Pa.) supplemented with 1% potassium ferricyanide (ThermoFisher). Eyes were dehydrated through a graded series of ethanol and embedded in Polybed 812 (Polysciences, Warrington, Pa.). Semi-thin (300 nm) sections were cut on a Reichart Ultracut, stained with 0.5% toluidine blue (ThermoFisher) and examined under a light microscope. Ultrathin sections (65 nm) were stained with uranyl acetate (Electron Microscopy Sciences) and Reynold's lead citrate (ThermoFisher). Sections were viewed on a JEOL JEM 1011 transmission electron microscope (JEOL, Peobody Mass.) at 80 KV. Images were taken using a side-mount AMT 2k digital camera (Advanced Microscopy Techniques, Danvers, Mass.).

### Results

Human ADSCs Differentiation into Trabecular Meshwork Cells

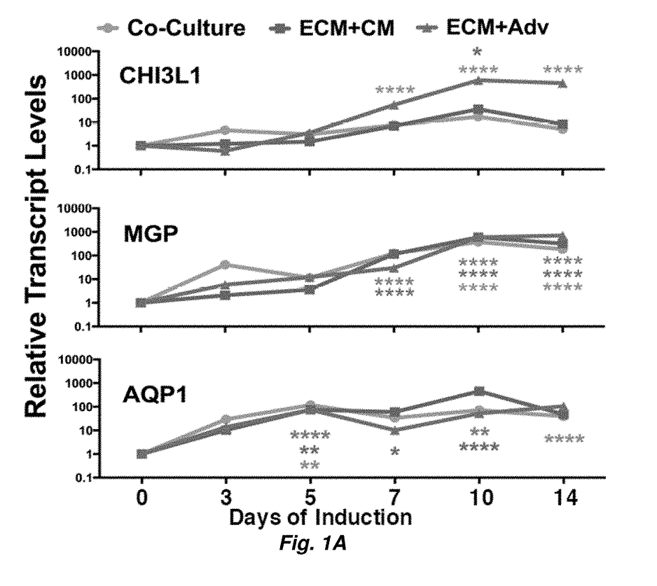

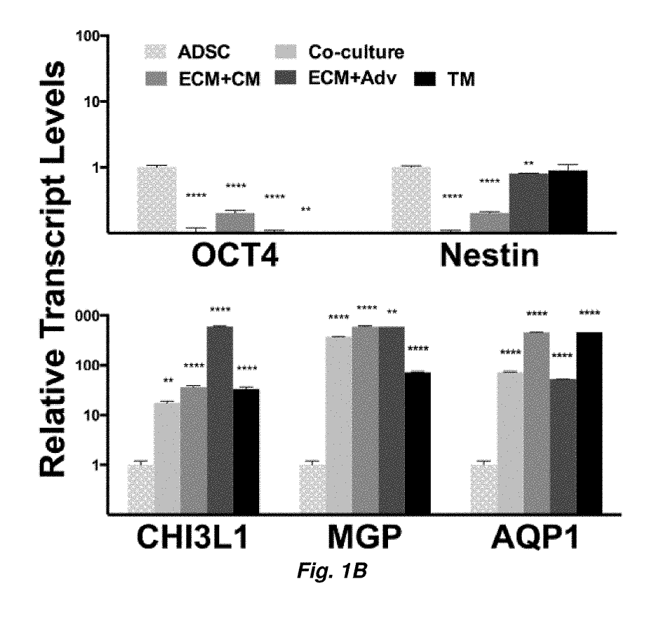

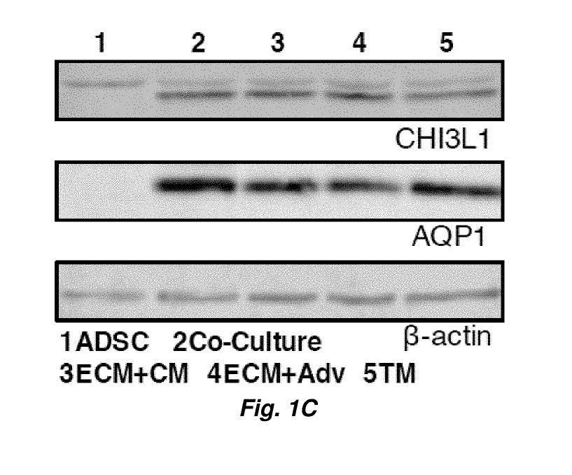

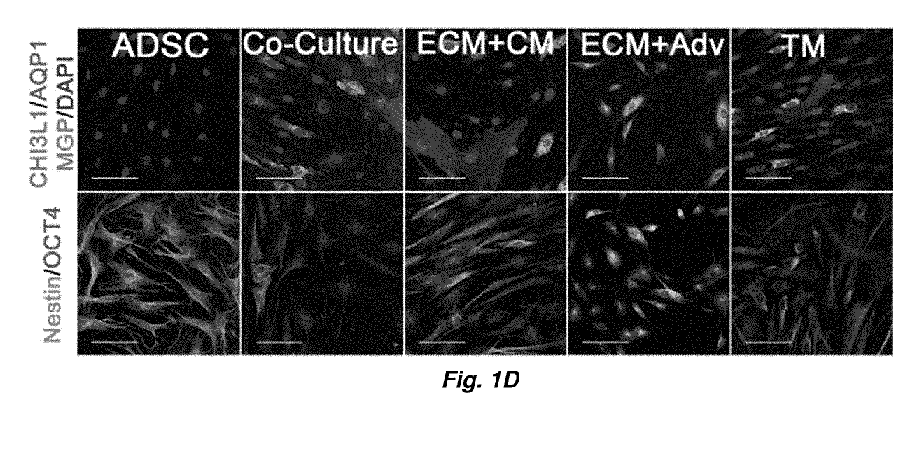

ADSC-TM cells were generated using three different methods: (1) co-culture with TM cells (Co-culture); (2) culture on TM-secreted extracellular matrix in TM-conditioned medium (ECM+CM); and (3) culture on TM-secreted ECM and Advanced MEM (ECM+Adv). To optimize the timescale of ADSC-TM derivation, quantitative real-time PCR was performed using RNA harvested from ADSCs from day 0 through day 14 of induction. The expression of TM cell markers such as CHI3L1, AQP1 and MGP increased in ADSCs after induction, comparable to primary TM cells (FIG. 1A), effectively reaching a maximum at Day 10. Thus, a 10-day induction period was used for all further ADSC-TM experiments.

FIG. 1B shows relative transcript levels of selected messenger RNAs in primary ADSCs, differentiated ADSC-TM cells and primary TM cells by qPCR. Ten days after induction, the levels of stem cell markers OCT4 and nestin was reduced and those of TM cell markers CHI3L1, MGP, AQP1 was increased, similar to primary TM cells. Western blot results (FIG. 1C) show increased protein expression of CHI3L1 and AQP1 in ADSC-TM cells, displaying a pattern similar to human TM cells, but not ADSCs. This is consistent with immunofluorescent staining showing that ADSC-TM cells expressed CHI3L1, MGP, and AQP1, similar to primary TM cells (FIG. 1D. In addition, differentiated ADSC-TM cells still expressed stem cell markers OCT4 and nestin by immunostaining (FIG. 1D).

It has long been established that the outer portion of the TM layers are phagocytic and are thought to function as a pre-filter, removing debris from the aqueous humor. Hence, we examined whether induced ADSC-TM cells would possess phagocytic function after induction. After incubating with fluorescent S. aureus bioparticles, most ADSC-TM cells and primary TM cells (positive control) contained S. aureus bioparticles (FIG. 2A, top row) with a circular peri-nuclei distribution indicating a cytoplasmic location of the ingested bioparticles (FIG. 2A, bottom row). In contrast, most ADSCs did not ingest the bioparticles. Quantitative assessment (FIG. 2B) demonstrated phagocytosis in 86±12% ADSC-TM cells in the Co-culture group, comparable to that of human TM cells (93±3%, p>0.05). Only 65±13% cells in the ECM+CM group and 67±5% cells in the ECM+Adv group contained phagocytosed bioparticles, yet both groups had significantly higher phagocytosis rates than primary ADSC cells (***p<0.001).

### Response of ADSC-TM Cells to Dexamethasone Stimulation

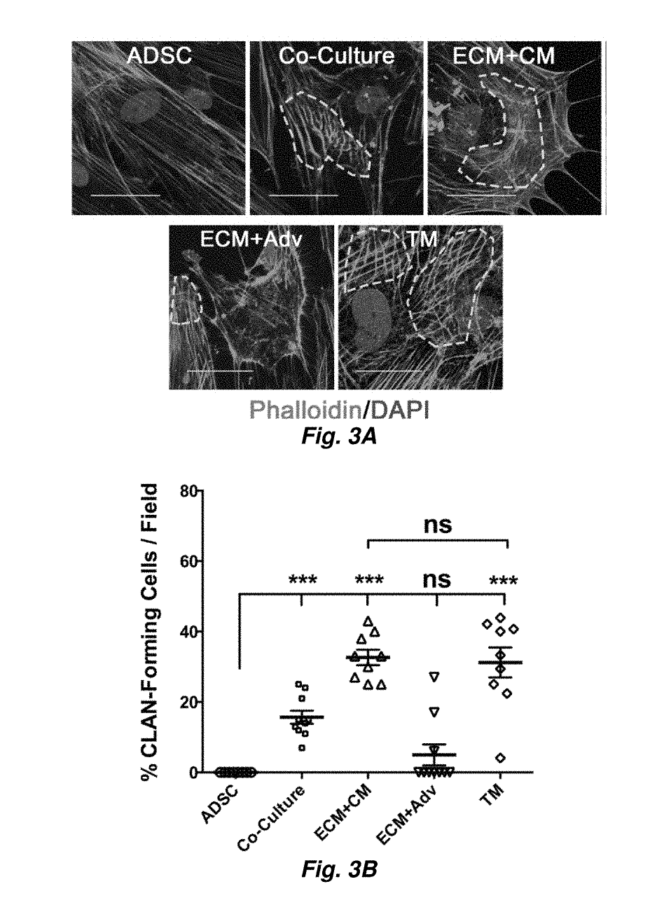

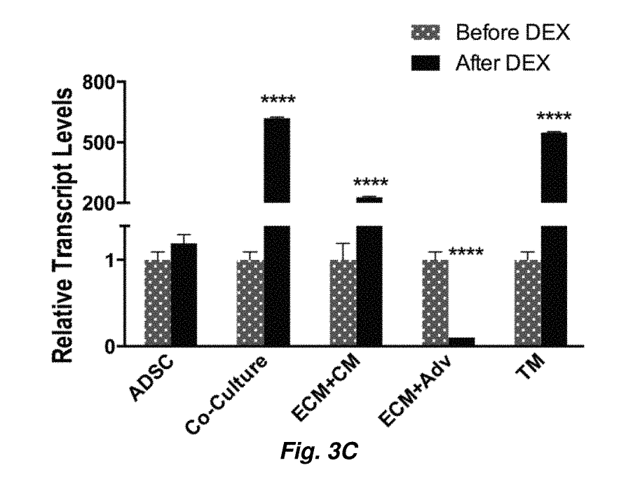

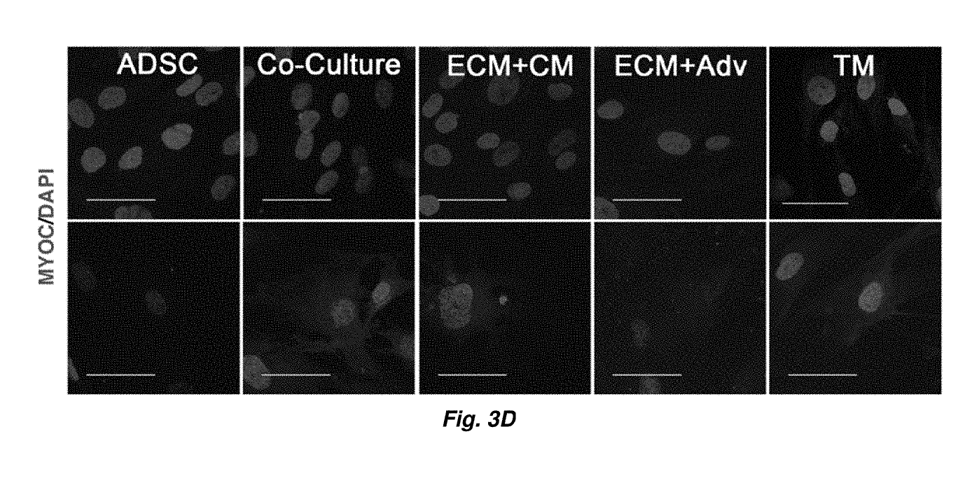

Clinically, topical administration of glucocorticoids to the eye can lead to development of ocular hypertension. Dexamethasone (DEX) treatment induces various changes in cultured TM cells and their secreted ECM, and is widely used in TM research to identify TM cell characteristics. After 10 days of induction, 200 nM DEX was applied to ADSC-TM cells for 7 days. A well-characterized response of TM cells to DEX is the formation of cross-linked actin networks (CLANs). By fluorescence microscopy, we observed that DEX caused a profound morphological change in the organization of microfilaments in TM cells as well as ADSC-TM cells, but not in primary ADSCs (FIG. 3A. The reorganized actin fibers found in TM cells resembled geodesic-dome-like polygonal lattices. Morphologically, CLANs in ADSC-TM cells from the Co-culture group were similar to those in the TM control group; other groups showed reorganization of microfilament fibers as well (FIG. 3A). The percentage of CLAN-forming cells was comparable between ADSC-TM cells in ECM+CM and primary TM cells (33±7% v.s. 31±0.05%, p>0.05) (FIG. 3B). Although the percentage of CLAN-forming cells was less in Co-culture induction (16±6%) than in the ECM+CM condition (33±7%), it was still significantly higher than primary ADSC cells in which no CLANs were observed. ECM+Adv induction did not produce many CLAN-forming cells (5±4%), a value which was not statistically different from that in primary ADSCs.

Another response of TM cells to DEX is upregulation of the glaucoma-associated gene myocilin (MYOC). Mutant MYOC is misfolded and accumulates intracellularly as soluble and insoluble aggregates, accompanied by endoplasmic reticulum (ER) stress. After DEX treatment, MYOC mRNA expression was significantly elevated in primary TM cells and ADSC-TM cells induced by co-culture or by ECM+CM (FIG. 3C). In comparison, MYOC mRNA expression was not elevated after DEX treatment in primary ADSCs and ADSC-TM cells induced in ECM+Adv (FIG. 3C). Immunofluorescent staining shows MYOC expression in TM cells and ADSC-TM cells after DEX treatment in all induction conditions but not in ECM+Adv nor in ADSCs (FIG. 3D. Although primary TM cells had weak expression of MYOC before DEX treatment, MYOC was mainly located in the perinuclear region after DEX treatment, which indicates that nonsecreted MYOC accumulated in the ER, which may be related to ER stress in TM cells. Previous work has shown ER proliferation in TM cells after DEX exposure, which supports this hypothesis (Wilson K, et al. Dexamethasone induced ultrastructural changes in cultured human trabecular meshwork cells. Curr Eye Res. 1993; 12(9):783-93).

### ADSC-TM Cell Transplantation In Vivo

For in vivo experiments, the induction condition of ECM+CM for ADSC-TM cells was selected. This induction efficiency is about the same as Co-Culture (FIGS. 1, 2, and 3) without the possibility of contamination with primary TM cells. Human ADSCs at passage 4 and ADSC-TM cells induced in ECM+CM for 10 days at passage 4 were injected into the anterior chamber of adult C57BL/6 wild type mice. Human fibroblasts and medium only (sham) were injected as controls. Age-matched WT mice served as normal controls. Cells were prelabeled with fluorescent green dye DiO as previously reported (31). 10,000 cells were transplanted in a volume of 2 μl of DMEM/F12. IOP was measured regularly and mice were sacrificed on Day 30 after transplantation. Eyes were enucleated and either perfused to measure outflow facility followed by dissection for RNA extraction and qPCR; or fixed to create cryosections for immunofluorescent staining and ultrathin sections for transmission electron microscopy (TEM). 29-32 mice in each group were included in the in vivo experiments.

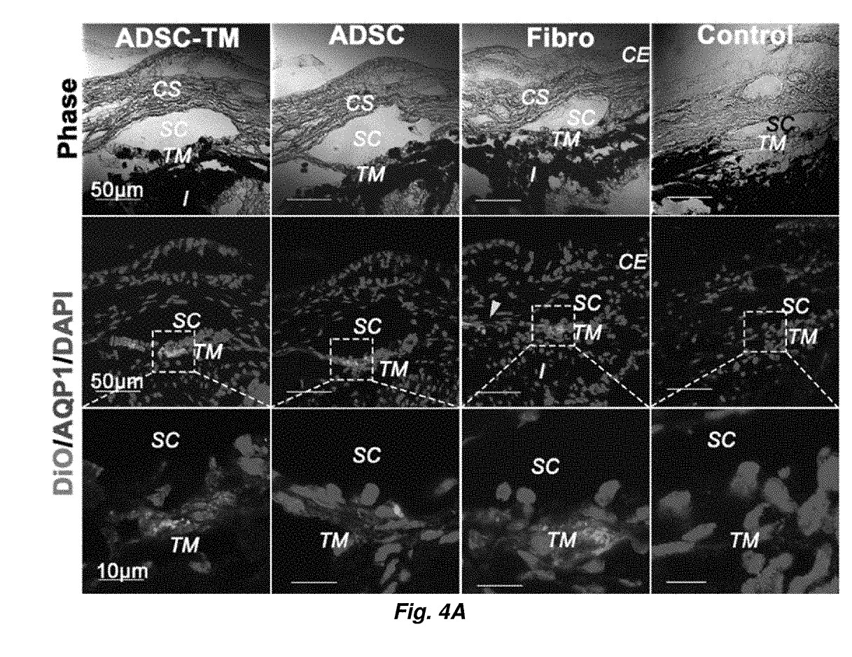

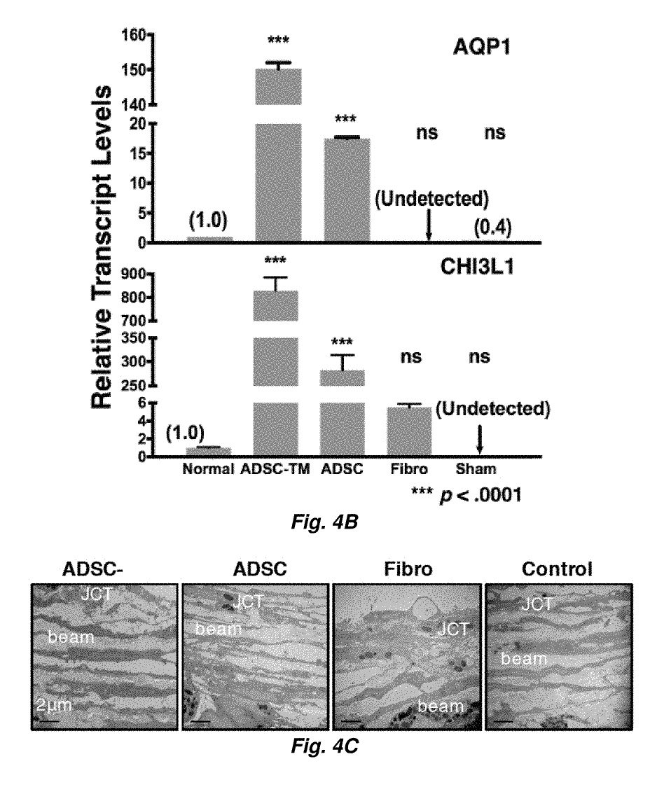

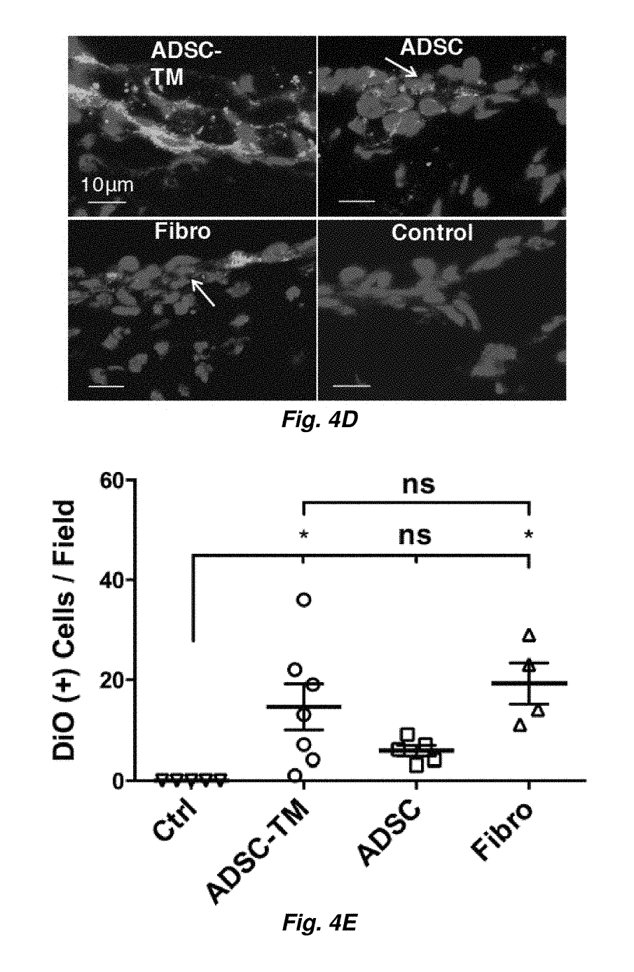

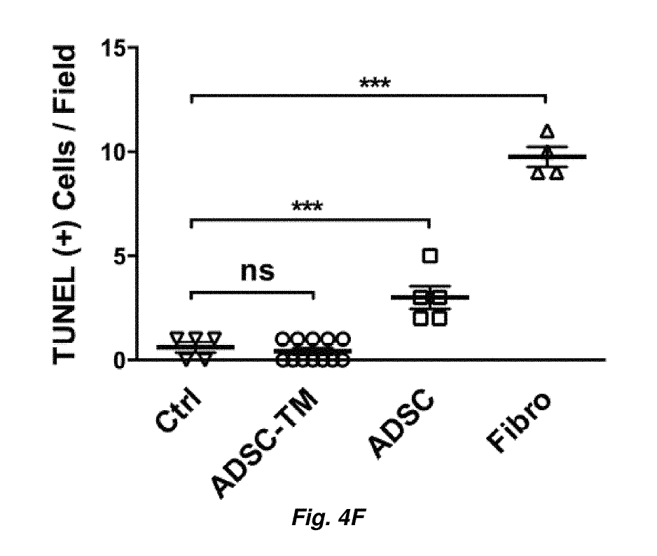

Immunofluorescent staining of mouse eye sagittal cryosections demonstrated that both injected ADSC-TM cells and ADSCs were present in the TM (FIG. 4A), distributed throughout the cell layers of the TM. It may indicate that the cells integrated into the TM tissue. Although green fibroblasts could also be seen in the TM, fibroblasts displayed off-target attachment into other tissues of anterior chamber, such as corneal endothelium (FIG. 4A, middle row, arrowhead) and iris. This suggests that these cells attached to tissues with low specificity, which could eventually cause disturbance to anterior chamber microstructure and aqueous humor dynamics.

More profoundly, integrated ADSC-TM cells expressed TM cell marker AQP1. AQP1 was detected in both injected green cells and endogenous mouse TM cells (FIG. 4A, bottom row). Although AQP1 is not a TM-specific marker, it is expressed in TM cells as well as in corneal stromal and endothelial cells. In contrast, injected fibroblasts were AQP1 negative. Consistent with image data shown above, qPCR results using human-specific primers (FIG. 4B, Table 3) showed dramatically larger messenger levels for the TM markers AQP1 and CHI3L1 in the ADSC-TM and ADSC injected groups. The expression level in the ADSC-TM group was higher than in the ADSC group, but the greater messenger levels in both ADSC-TM and ADSCs was statistically significant (****p<0.0001) compared to the fibroblast-injection, normal control and sham control groups.

The microstructure of the mouse TM region was examined by TEM (FIG. 4C). Similar to the normal control group, ADSC-TM and primary ADSC groups showed intact TM microstructure, with thin and well-defined extracellular beams covered by TM cells. In the fibroblast injection group, however, the extracellular beams were irregular and the JCT region is more compacted than the others.

### Viability of Transplanted ADSC-TM Cells

Although green fluorescent DiO stained cells remained visible in the tissue 1 month after transplantation, it was important to determine if the injected human cells survived in the mouse TM after xenotransplantation. The TUNEL assay was used to assess the viability of transplanted cells and any possible associated damage to endogenous host cells. DiO+green ADSCs, ADSC-TM cells and fibroblasts were present in the TM tissue (FIGS. 4A and 4D). Few of the green fluorescent ADSCs and fibroblasts exhibited TUNEL staining (FIG. 4D). A lot of non-green host mouse TM cells in the fibroblast injected TM tissue, however, were apoptotic, stained red (FIG. 4D).

To quantify the number of injected green cells in the TM region, we randomly selected 4-6 cryosections from 3 samples in each experimental group and counted the number of DiO positive cells per view in all experimental groups. The average cell numbers per section were: ADSCs (6±2), ADSC-TM cells (15±12), fibroblasts (19±8) (FIG. 4E). Although there were many injected green fibroblasts in the TM region or adjacent area, the counts of apoptotic cells for both green (injected exogenous cells) and non-green (endogenous cells) were largest in the fibroblast-injected TM tissue (FIG. 4F) among all the experimental groups. Statistical analysis on TUNEL staining indicated that the number of apoptotic cells for both green (injected exogenous cells) and non-green (endogenous cells) in the fibroblast injection group was significantly larger than in all the other groups (FIG. 4F). The apoptotic endogenous cells numbers in ADSC-TM injected eyes were comparable to those in control eyes (0.4±0.4, p>0.05). Primary ADSCs caused more apoptosis in endogenous mouse TM cells than in control eyes (3±1, p<0.05).

Aqueous Humor Dynamic Hemostasis Remains Intact after ADSC-TM Transplantation

To assess if the transplanted ADSC-TM cells could function to maintain intraocular pressure (TOP) and outflow facility within a normal range, we measured mouse IOP before transplantation and at 3 d, 5 d, 10 d, 2 wk, 3 wk and 1 month after anterior chamber injection (FIG. 5A). Each cell injection group was compared to the uninjected control group. On day 3 after transplantation, IOP elevation was observed in ADSC-TM (17.8±3.3 mmHg, **p<0.01), ADSC (18.1±5.7 mmHg, *p<0.05) and fibroblast (19.4±4.9 mmHg, ****p<0.0001) injection groups compared with the control group (15.2±3.3 mmHg). From day 5, the IOP in ADSC and ADSC-TM injection groups reduced and was maintained in the normal range, comparable to controls. In contrast, the IOP in fibroblast-injected eyes remained elevated up to 1 month post-transplantation (19.1±2.5 mmHg vs. 14.9±2.5 mmHg, ****p<0.0001).

Aqueous humor outflow facility is inversely proportional to the fluid flow resistance of the conventional outflow pathway. Ex vivo mouse eye perfusion followed the procedures described by Lei et al (51). Outflow facility in ADSC-TM injected eyes (FIG. 5C) was not different from that of the control group (FIG. 5F) (0.020±0.002 μl·min⁻¹·mmHg⁻¹ vs. 0.020±0.002 μl·min⁻¹·mmHg⁻¹, p=0.95). Similarly, there was no significant difference in outflow facility between primary ADSC injection (FIG. 5D) and control groups (FIG. 5F) (0.021±0.002 μl·min⁻¹·mmHg⁻¹ vs. 0.020±0.002 μl·min⁻¹·mmHg⁻¹, p=0.6503). In contrast, the fibroblast injection group (FIG. 5E) had reduced outflow facility at 0.011±0.040 μl·min⁻¹·mmHg⁻¹, significantly lower than that of control (FIG. 5F) (p=0.01). The outflow facilities of all conditions were shown together in FIG. 5B.

Here, it is shown that human ADSCs can be induced in vitro to differentiate into phagocytic TM cells expressing the TM cell markers CHI3L1, AQP1 and MGP. The induced cells (ADSC-TM) are responsive to dexamethasone stimulation with increased expression of MYOC and formation of CLANs within the cytoplasm. After intracameral injection into wild type mice, both ADSC-TM cells and primary ADSCs can specifically home and integrate to the TM tissue expressing TM cell markers CHI3L1 and AQP1. The xenotransplantation did not adversely affect the IOP and outflow facility in normal eyes for up to 30 days after injection. The chemokine CXCR4 and its ligand SDF1 may play an important role in ADSC-TM cell homing to the TM tissue. In contrast, injected fibroblasts attached to the TM tissue, corneal endothelium and iris in the anterior chamber, suggesting that ADSCs and ADSC-TM cells actively home to the TM tissue while fibroblasts passively attach to the tissues. This study is novel and shows that it is feasible to apply ADSCs as a candidate for TM autologous cell transplantation and regeneration strategy for POAG patients.

Previous work showed that human trabecular meshwork stem cells can home and integrate into normal mouse TM tissue and maintain the mouse IOP in normal range (Du Y, et al. Stem cells from trabecular meshwork home to TM tissue in vivo. Invest Ophthalmol Vis Sci. 2013; 54(2):1450-9), opening the door to explore stem cell-based therapy for glaucoma. On the other hand, it is not easy to use autologous TM stem cells for glaucoma treatment since cell harvesting would require ocular microsurgery and TM stem cells may be depleted or dysfunctional in POAG patients. ADSCs have advantages over other types of stem cell candidates, in that they can be harvested in large amounts with minimally invasive approaches. In this study, we showed that ADSCs can be induced to differentiate into TM-like cells by co-culturing with TM cells or by culturing them on TM-secreted ECM for only 10 days. Both approaches yielded differentiated TM-like cells as judged by phagocytosis and response to DEX stimulation. A previously published study reported that ˜34% of human TM cells formed CLANs after DEX exposure (52). Our results showed a similar percentage of cells forming CLANs, including primary TM cells and ADSC-TM cells induced by ECM+CM and by co-culture. The ADSC-TM cells induced in ECM+Adv had fewer CLAN-forming cells, and we therefore eliminated this condition as an induction protocol. For DEX stimulation, we treated cells with 200 nM DEX for 7 days which is different from others using 100 nM (45, 50, 52). Our primary ADSCs were cultured initially with 100 nM DEX to reduce possible fibroblast contamination (53) and DEX was removed from passage 2 to 3. For DEX stimulation experiments, we used 200 nM DEX on all the cell types so ADSCs could still be stimulated by DEX.

Human ADSC-TM cells transplanted into mouse anterior chamber survived for up to 1 month (FIGS. 4A-4F) and maintained IOP and outflow facility within normal ranges (FIGS. 5A-5D). The integrated ADSC-TM cells expressed the TM cell markers CHI3L1 and AQP1. Interestingly, primary ADSC cells, which intrinsically lack TM marker expression, could be induced to become TM-like cells in vivo expressing TM cell markers CHI3L1 and AQP1, suggesting that the local environment of mouse TM can induce human primary ADSC cells to differentiate into TM-like cells in vivo. In contrast, even though some fibroblasts seemed to integrate into the TM tissue, they did not express any TM-related markers and they elevated mouse IOP after injection. Therefore, we suggest that the fibroblasts were not truly integrated to the TM but passively followed the aqueous outflow to reside to the TM. The endogenous TM cells only had very weak to none expression of AQP1 by immunofluorescent staining (FIG. 4A). It may indicate that fibroblasts themselves or the inflammatory response they caused may affect the characteristics of residual TM cells and their function.

On day 3 after transplantation, all groups with cell injection had increased IOP, which then declines to the normal range all cell injection groups except the fibroblast-injection group, which had elevated IOP for up to 1 month. The transient IOP elevation may be due to a temporary inflammatory response after xenotransplantation of human cells. We previously showed that after transplantation of human cells into mouse corneal stromal, transient inflammatory cells migrated from mouse bone marrow to the corneal stromal at 24 hours and reduced dramatically at 72 hours (Du Y, et al. Stem cell therapy restores transparency to defective murine corneas. Stem Cells. 2009; 27(7):1635-42). There were no inflammatory cells at 2 weeks after stem cell transplantation, but inflammatory cells persisted in the corneas with fibroblast injection. This may explain why the IOP remained elevated on the eyes with fibroblast injection.

ADSC-TMs transplantation maintained aqueous humor hemostasis with normal IOP and outflow facility. Moreover, transmission electron microscopy indicated that both ADSC-TM cells and ADSCs injected into the anterior chamber maintained normal microstructure of the TM with normal cell morphology and ECM formation. These findings suggest that ADSC-TMs may be a safe cell therapy resource for POAG.

The outflow of aqueous humor facilitates stem cell delivery from the anterior chamber into the TM. However, it is interesting that fibroblast implantation did not exclusively follow this route; instead, fibroblasts also attached into anterior segment structures such as iris and corneal endothelium. This observation led us to further investigate the integration of ADSC-TM cells into TM tissue. In vitro experiments showed that the CXCR4/SDF1 axis—an essential pathway for controlling the navigation of progenitors between the bone marrow and blood—plays a role in chemotaxis and affinity between ADSC-TM cells and TM cells, whereas no such phenomenon was observed between primary ADSCs and TM cells, consistent with a previous report that CXCR4 and its ligand SDF1 are likely not major homing factors for ADSCs (Albersen M, et al. Expression of a Distinct Set of Chemokine Receptors in Adipose Tissue-Derived Stem Cells is Responsible for In Vitro Migration Toward Chemokines Appearing in the Major Pelvic Ganglion Following Cavernous Nerve Injury. Sex Med. 2013; 1(1):3-15). Additional pathways might be examined to thoroughly understand the mechanism(s) of specific homing of ADSCs and ADSC-TM cells. For example, a conditioned SDF1 knockdown mouse model could be generated to confirm the effect of the CXCR4-SDF1 axis on ADSC-TM cell homing in TM regeneration. Further, CXCR7 or other receptors may be involved in ADSC homing to the TM tissue and this area requires further exploration. Understanding such homing mechanism(s) is a promising tool to manipulate these pathways for enhanced homing and integration of implanted stem cells and consequently better restoration of TM function.

In conclusion, these results provide evidence that ADSCs can be induced to functional TM cells and can be an attractive autologous stem cell resource for TM restoration as a stem cell-based therapy for glaucoma.

The following clauses present various illustrative aspects of the present invention:

1. A method of producing functional trabecular meshwork (TM) cells, comprising culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.
2. The method of clause 1, in which the ADSCs are cultured on decellularized trabecular meshwork.
3. The method of clause 2, wherein the decellularized trabecular meshwork is prepared by culturing TM cells or functional TM cells in a cell culture vessel; and decellularizing the cultured TM cells.
4. The method of clause 2, wherein the TM cells are cultured to confluence before decellularization.
5. The method of clause 3 or clause 4, wherein the cultured TM cells are decellularized by exposure to ammonium hydroxide.
6. The method of clause 1 or clause 2, wherein the ADSCs are cultured in TM cell-conditioned medium or functional TM cell-conditioned medium.
7. The method of clause 1, in which the functional TM cells:
a. express TM cell markers CHI3L1, MGP and AQP1;
b. exhibit decreased expression of nestin as compared to ADSCs;
c. ingest inactivated S. aureus particles in the manner of primary TM cells; and/or
d. exhibit increased MYOC expression in response to dexamethasone treatment.
8. The method of any of clauses 1-8, wherein the ADSCs are cultured with primary trabecular meshwork cells, extracellular matrix produced by trabecular meshwork cells, and/or trabecular meshwork cell-conditioned medium for at least 2 days.
9. The method of clause 8, wherein the ADSCs are cultured with primary trabecular meshwork cells, extracellular matrix produced by trabecular meshwork cells, and/or trabecular meshwork cell-conditioned medium for from 2 to 14 days.
10. The method of any of clauses 1-9, in which the ADSCs are expanded ex vivo.
11. A method of treating glaucoma in a patient in need thereof, comprising introducing, e.g. injecting or placing, into an eye of the patient a functional trabecular meshwork (TM) cell prepared by differentiation of an adipose-derived stem cell (ADSC).
12. The method of clause 11, wherein the functional TM cell is introduced, e.g. injected or placed, into the anterior chamber of the patient's eye.
13. The method of clause 11 or 12, wherein the functional TM cell is prepared culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.
14. The method of clause 11 or 12, wherein the functional TM cell is prepared by a method of any one of clauses 1-10.
15. The method of any of clauses 11-14, wherein the ADSC is autologous to the patient.
16. A method of implanting trabecular meshwork (TM) cells in an eye of a patient in need thereof, comprising introducing, e.g. injecting or placing, into the eye of the patient an adipose-derived stem cell (ADSC) or a functional TM cell prepared from an ADSC.
17. The method of clause 16, wherein a functional TM cell prepared from an ADSC is introduced, e.g. injected or placed, into the patient's eye and the functional TM cell is prepared culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.
18. The method of clause 17, wherein a functional TM cell prepared from an ADSC is introduced, e.g. injected or placed, into the patient's eye and the functional TM cell is prepared according to the method of any of clauses 1-10.
19. The method of clause 16, comprising introducing, e.g. injecting or placing, an ADSC into the patient's eye.
20. The method of any of clauses 16-19, wherein the ADSC or functional TM cell is introduced, e.g. injected or placed, into the anterior chamber of the patient's eye.
21. The method of any of clauses 16-20, wherein the ADSC is autologous to the patient.
22. A method of repairing or regenerating the aqueous outflow pathway of an eye of a patient, comprising introducing, e.g. injecting or placing, into an eye of the patient an adipose-derived stem cell (ADSC) or a functional TM cell prepared from an ADSC.
23. The method of clause 22, wherein a functional TM cell prepared from an ADSC is introduced, e.g. injected or placed, into the patient's eye and the functional TM cell is prepared culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.
24. The method of clause 22, wherein a functional TM cell prepared from an ADSC is introduced, e.g. injected or placed, into the patient's eye and the functional TM cell is prepared according to the method of any of clauses 1-10.
25. The method of clause 22, comprising introducing, e.g. injecting or placing, an ADSC into the patient's eye.
26. The method of any of clauses 22-25, wherein the ADSC or functional TM cell is introduced, e.g. injected or placed, into the anterior chamber of the patient's eye.

While the present invention is described with reference to several distinct embodiments, those skilled in the art may make modifications and alterations without departing from the scope and spirit. Accordingly, the above detailed description is intended to be illustrative rather than restrictive.

We claim:

1. A method of producing functional trabecular meshwork (TM) cells, comprising culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.

2. The method of claim 1, in which the ADSCs are cultured on decellularized trabecular meshwork.

3. The method of claim 2, wherein the decellularized trabecular meshwork is prepared by culturing TM cells or functional TM cells in a cell culture vessel; and decellularizing the cultured TM cells.

4. The method of claim 2, wherein the TM cells are cultured to confluence before decellularization.

5. The method of claim 1, wherein the ADSCs are cultured in TM cell-conditioned medium or functional TM cell-conditioned medium.

6. The method of claim 1, in which the functional TM cells:
a. express TM cell markers CHI3L1 and AQP1;
b. exhibit decreased expression of nestin as compared to ADSCs;
c. ingest inactivated S. aureus particles in the manner of primary TM cells; and/or
d. exhibit increased MYOC expression in response to dexamethasone treatment.

7. The method of claim 1, wherein the ADSCs are cultured with primary trabecular meshwork cells, extracellular matrix produced by trabecular meshwork cells, and/or trabecular meshwork cell-conditioned medium for at least 2 days.

8. The method of claim 7, wherein the ADSCs are cultured with primary trabecular meshwork cells, extracellular matrix produced by trabecular meshwork cells, and/or trabecular meshwork cell-conditioned medium for from 2 to 14 days.

9. A method of treating glaucoma in a patient in need thereof, comprising introducing into an eye of the patient an adipose-derived stem cell (ADSC), or a functional trabecular meshwork (TM) cell prepared by differentiation of an ADSC.

10. The method of claim 9, wherein the ADSC or functional TM cell is introduced into the anterior chamber of the patient's eye.

11. The method of claim 9, wherein a functional trabecular meshwork (TM) cell prepared by differentiation of an ADSC is introduced into the eye and the functional TM cell is prepared culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.

12. The method of claim 1, wherein the ADSC is autologous to the patient.

13. A method of implanting trabecular meshwork (TM) cells in an eye of a patient in need thereof, comprising introducing into the eye of the patient an adipose-derived stem cell (ADSC) or a functional TM cell prepared from an ADSC.

14. The method of claim 13, wherein the patient has glaucoma.

15. The method of claim 13, wherein a functional TM cell prepared from an ADSC is introduced into the patient's eye and the functional TM cell is prepared culturing adipose-derived stem cells (ADSCs) with primary trabecular meshwork cells, extracellular matrix produced by trabecular meshwork cells, and/or trabecular meshwork cell-conditioned medium, for a time sufficient to cause differentiation of the ADSCs to functional TM cells.

16. The method of claim 15, wherein a functional TM cell prepared from an ADSC is injected into the patient's eye and the functional TM cell is prepared according to the method of claim 1.

17. The method of claim 13, comprising introducing an ADSC into the patient's eye.

18. The method of claim 13, wherein the ADSC or functional TM cell is introduced into the anterior chamber of the patient's eye.

19. The method of claim 13, wherein the ADSC is autologous to the patient.


SEQUENCE LISTING (14 sequences)

SEQ ID NO: 1 - DNA, 23 bp, Artificial Sequence
  18S rRNA forward PCR primer
ccctgtaattggaatgagtccac

SEQ ID NO: 2 - DNA, 18 bp, Artificial Sequence
  18S rRNA reverse PCR primer
gctggaattaccgcggct

SEQ ID NO: 3 - DNA, 20 bp, Artificial Sequence
  CHI3L1 forward PCR primer
gatgtgacgctctacggcat

SEQ ID NO: 4 - DNA, 20 bp, Artificial Sequence
  CHI3L1 Reverse PCR primer
tgatgaaagtccggcgactc

SEQ ID NO: 5 - DNA, 20 bp, Artificial Sequence
  MGP forward PCR primer
gccgccttagcggtagtaac

SEQ ID NO: 6 - DNA, 20 bp, Artificial Sequence
  MGP reverse PCR primer
tctctgctgaggggatatga

SEQ ID NO: 7 - DNA, 20 bp, Artificial Sequence
  AQP1 forward PCR primer
ctgcacaggcttgctgtatg

SEQ ID NO: 8 - DNA, 20 bp, Artificial Sequence
  AQP1 reverse PCR primer
tgttccttgggctgcaacta

SEQ ID NO: 9 - DNA, 20 bp, Artificial Sequence
  Myocilin forward PCR primer
aagcccacctacccctacac

SEQ ID NO: 10 - DNA, 20 bp, Artificial Sequence
  Myocilin reverse PCR primer
tccagtggcctaggcagtat

SEQ ID NO: 11 - DNA, 20 bp, Artificial Sequence
  OCT4 forward PCR primer
gtggaggaagctgacaacaa

SEQ ID NO: 12 - DNA, 20 bp, Artificial Sequence
  OCT4 reverse PCR primer
ggttctcgatactggttcgc

SEQ ID NO: 13 - DNA, 19 bp, Artificial Sequence
  Nestin forward PCR primer
aagatgtccctcagcctgg

SEQ ID NO: 14 - DNA, 20 bp, Artificial Sequence
  Nestin reverse PCR primer
gagggaagtcttggagccac